(19) United States
(12) Patent Application Publication
(10) Pub. No.: US 2001/0027334 A1
(43) Pub. Date: Oct. 4, 2001

(54) Body-cooling container

(76) Inventor: Anthony Dale White, Bowling Green, KY (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/779,883

(22) Filed: Feb. 8, 2001

Related U.S. Application Data
(60) Provisional application No. 60/181,239, filed on Feb. 8, 2000.

(51) Int. Cl. A61F 7/00

(52) U.S. Cl. 607/108; 607/114

(57) ABSTRACT

A cooling container has a pillow-like portion with a chamber for receiving ice or another cooling medium, and a connected pad-like portion having passages and a reservoir for receiving water from the ice. The chamber has a sufficiently large opening through which all of the ice or other cooling medium to be placed in the chamber can be substantially contemporaneously passed into the chamber via the opening. In one example of use, the pillow-like portion cools a user's head and neck, and the pad-like portion cools the user's upper torso.

### CROSS-REFERENCE TO RELATED APPLICATION

This patent application claims the benefit of U.S. Provisional Patent Application No. 60/181,239, which was filed Feb. 8, 2000, and is incorporated herein by reference.

### FIELD OF THE INVENTION

The present invention pertains to containers for receiving cool substances and being placed in contact with a user for cooling purposes and, more particularly, to such cooling containers that are used while sunbathing or during any other activity that might require cooling.

### BACKGROUND OF THE INVENTION

It is common for people in warm environments to cool themselves by placing containers of cool substances against their bodies. For example, U.S. Pat. No. 5,916,088 discloses a pillow/headrest for keeping a user cool and comfortable while sunbathing. The pillow includes a slot for receiving a freezer pack of frozen material so that the pillow can be used to keep the user's head and neck cool. Unfortunately, the cooling effect of this pillow is limited, because while the cooling pillow is in contact with the user's head and neck, it will not be in contact with any or a significant portion of the user's torso. In contrast, U.S. Pat. No. 4,017,921 discloses a cooling blanket having multiple elongated chambers adapted for each receiving ice. Although blankets are often sized for interacting with a substantial portion of the user's body, the disclosed cooling blanket disadvantageously requires that ice cubes be manually filled into each of its chambers individually, and each of the chambers must be individually unzipped and zipped as part of the filling process. The use of this cooling blanket can be disadvantageously labor intensive since each chamber must be individually filled and sealed.

Accordingly, there is a need in the art for an improved device for cooling a user that can be easily used and is capable of cooling a substantial portion of the user's body.

### SUMMARY OF THE INVENTION

In accordance with one aspect, the present invention solves the above and other problems by providing a cooling container having a pillow-like portion having a chamber for receiving ice or another cooling medium, and a connected pad-like portion, preferably in the form of passages and a reservoir, for receiving cool water from the ice, with the ice preferably remaining in the chamber. In accordance with this aspect, the chamber has a sufficiently large opening through which all of the ice or other cooling medium to be placed in the chamber can be substantially contemporaneously passed into the chamber. Accordingly, the cooling container of the present invention can be quickly and easily loaded with ice, or the like. In one example of use and depending upon the size of the cooling container, the pillow-like portion cools a user's head and neck, and the pad-like portion cools at least the upper torso of the same user. In accordance with this example, the cooling device advantageously can be used to cool a substantial portion of the user's body.

In accordance with one aspect of the present invention, the cooling container has a body constructed of flexible and water-impermeable material, defining an interior space and an opening to the interior space through which the ice can be introduced into the interior space, and including walls that are positioned on opposite sides of the interior space. Portions of the walls are connected to define at least one obstruction positioned in the interior space for partially separating first and second sections of the interior space. In one example of this aspect, the first and second sections respectively correspond to the pillow-like and pad-like portions described above, and the obstruction(s) function to hold the ice in the pillow-like section. That is, the first section is adjacent the opening for receiving the ice, and the first section and the second section are connected so that water resulting from the ice can flow from the first section to the second section. Also in accordance with this aspect, a fastening device is provided for closing the opening so that the interior space of the cooling container can be substantially sealed.

In accordance with one aspect of the present invention, the walls are sheets, such as polymeric laminates, that are connected, such as by fusing, to form the cooling container. More specifically regarding this aspect, each sheet has a margin extending around the periphery of the sheet and the margin includes first and second sections. The sheets are arranged one on top of the other, and then the first section of the margin of one of the sheets is connected to the first section of the margin of the other of the sheets, so that the interior space is defined between nonmarginal portions of the sheets. The opening to the interior space is defined between the second sections of the margins of the sheets. At least one nonmarginal portion of one of the sheets is connected to at least one nonmarginal portion of the other sheet to define the obstruction positioned in the interior space, for at least partially separating the first and second sections of the interior space.

In accordance with one aspect of the present invention, the fastening device is a zipper-type fastening device. That is, a first part of the fastening device includes an elongated ridge and a second part of the fastening device includes an elongated groove. According to this aspect, the parts of the fastening device are mated and releasably interlocked by forcing the ridge into the groove.

In accordance with one aspect of the present invention, the cooling container has opposite ends and a length is defined between the opposite ends, and the opening, which is for receiving ice or the like, includes opposite ends and a length is defined between the opposite ends of the opening. In accordance with this aspect, the length of the opening is advantageously at least about one third of the length of the cooling container, so that the chamber has a sufficiently large opening through which all of the ice, or other cooling medium, to be placed in the chamber can be substantially contemporaneously passed into the chamber.

In accordance with one aspect of the present invention, there are a plurality of obstructions spaced apart from one another in the interior space of the cooling container for at least partially separating the interior space of the cooling container into two or more sections. Preferably the obstructions are elongated and define a plurality of passages. In one example, each of the passages has opposite first and second ends that are respectively open to different sections within the internal space of the cooling container.

In accordance with one aspect of the present invention, the cooling container has one or more handles connected to or formed in opposite ends of the cooling container. In accordance with one example, where the cooling container is formed of sheets that are fused together in places, the sheets are fused together in an area and a handle hole is defined through the fused area such that the hole is encircled by fused together portions of the sheets.

In accordance with one aspect of the present invention, the cooling container is connected to a chair, such as by straps extending through attachment holes in the cooling container, or by straps that form a handle of the cooling container.

In accordance with one aspect of the present invention, the cooling container can be grasped by its handles and advantageously carried like a bag.

Aspects and advantages of the present invention in addition to those described above will be apparent to those of ordinary skill in the art upon studying the remainder of this disclosure.

### BRIEF DESCRIPTION OF THE DRAWINGS

Having thus described the invention in general terms, reference will now be made to the accompanying drawings, which are not necessarily drawn to scale, and wherein:

FIG. 1 is a top plan view of a cooling container in a flattened and horizontal configuration, in accordance with a first embodiment of the present invention;

FIG. 2 is a schematic side elevational view of the cooling container of FIG. 1 in its empty and flattened configuration;

FIG. 3 is a schematic cross-sectional view of the cooling container taken substantially along line 3-3 of FIG. 1, with the cooling container in a substantially filled configuration but the filling medium not shown;

FIG. 4 is a schematic cross-sectional view of the cooling container taken along line 4-4 of FIG. 1, with the cooling container in a substantially filled configuration but the filling medium not shown;

FIG. 5 is a schematic cross-sectional view of the cooling container taken along line 5-5 of FIG. 1, with the cooling container in a substantially filled configuration but with the filling medium not shown;

FIG. 6 is a partial view that schematically illustrates a bag of ice being inserted into the opening of the cooling container, in accordance with the first embodiment of the present invention;

FIG. 7 is a partial view that schematically illustrates the cooling container partially fastened to the back of a reclining chair, in accordance with the first embodiment of the present invention;

FIG. 8 schematically illustrates a rear side of a cooling container in a flattened configuration, in accordance with a second embodiment of the present invention;

FIG. 9 schematically illustrates the front side of the cooling container of FIG. 8;

FIG. 10 is a partial and schematic view illustrating a cover that is attached to the cooling container, with most of the cooling container being hidden from view behind the cover and illustrated by broken lines, and with the handle of the cooling container extending through openings in the cover and around a rung of a chair, in accordance with the second embodiment of the present invention;

FIG. 11 is a schematic perspective view illustrating the cooling container in an at least partially filled configuration, in accordance with the second embodiment of the present invention;

FIG. 12 is a schematic side cross-sectional view of the partially filled cooling container of FIG. 11; and

FIG. 13 is a view like FIG. 1, except that a cooling container of a third embodiment of the present invention is shown.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Generally described and in accordance with one aspect, the present invention is a cooling container having a pillow-like portion with a chamber for receiving and holding ice, and a pad-like portion preferably in the form of passages and a reservoir within which water from the ice melting in the chamber flows and collects. The cooling container can be placed upon any surface or structure upon which the user intends to sit or lie, and the user sits or lies on the cooling container for cooling purposes. For example, the cooling container can be hooked onto an upper portion of the backrest of a chair, and the user sits in the chair so that the pillow portion cools the user's head and neck and the pad portion cools the user's upper torso. In another example, the cooling container is smaller, and the pad portion is placed in the seat of a chair and the pillow portion is positioned as a back support. The cooling container can be used in any other manner. If desired for maximizing comfort or modifying the cooling effect of the cooling container, the cooling container can be covered with a cover or towel.

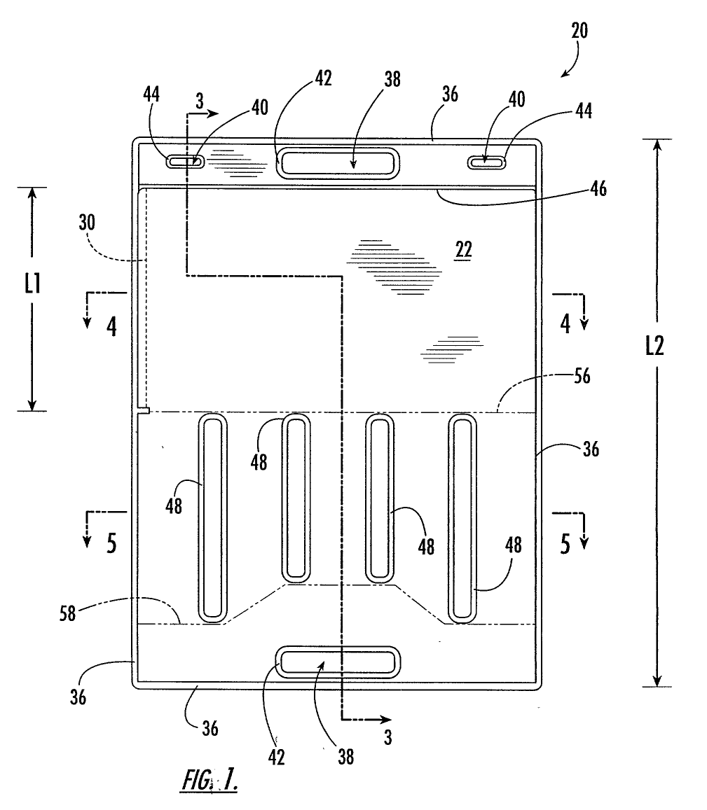

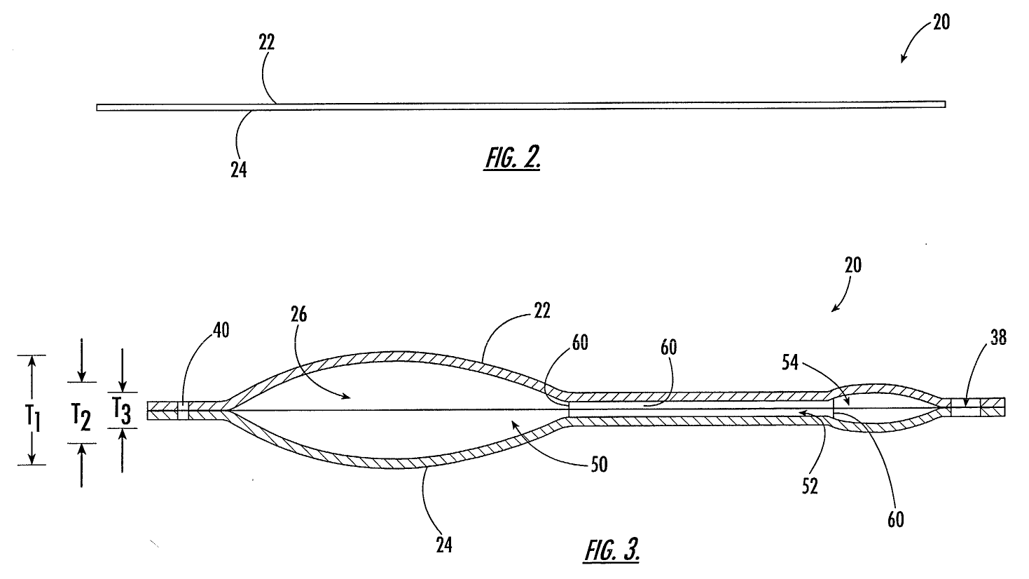

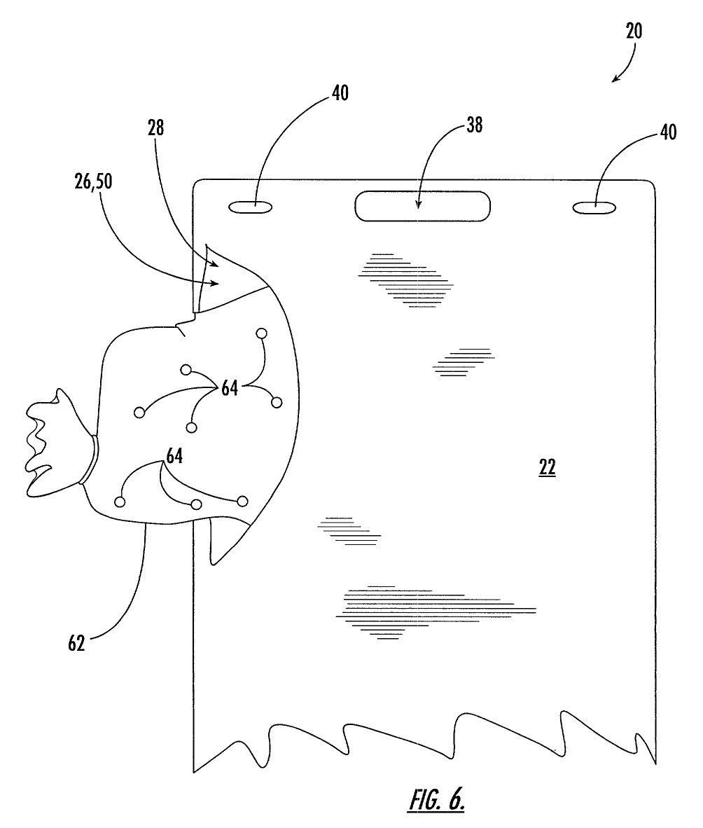

A first embodiment of the present invention will now be described with reference to FIGS. 1-7. Referring to FIG. 1, a cooling container according to the first embodiment is designated by the numeral 20. The cooling container 20 is constructed from two initially separate sheets 22 and 24 (also see FIGS. 2-5) which form the opposite walls of the cooling container. One of the sheets 24 is hidden from view behind the other of the sheets 22 in FIG. 1. In accordance with the first embodiment, each of the sheets 22 and 24 is a water-impermeable polymeric laminate, preferably with the laminate formed by bonding a polymeric film to a fabric. More specifically, each laminate or sheet 22, 24 is most preferably nylon coated polyvinylchloride.

During manufacturing, the sheets 22 and 24 are placed one on top of the other so that the non-fabric sides of the sheets face and abut one another, and then portions of the sheets are bonded together and portions are cut away to form the cooling container 20. In accordance with the first embodiment, the bonding is preferably carried by acoustically fusing (e.g., RF welding) the respective portions of the sheets 22 and 24 together. Alternatively, thermal energy can be used to fuse the respective portions of the sheets 22 and 24 together, or an adhesive or other means can be used to bond the respective portions of the sheets together.

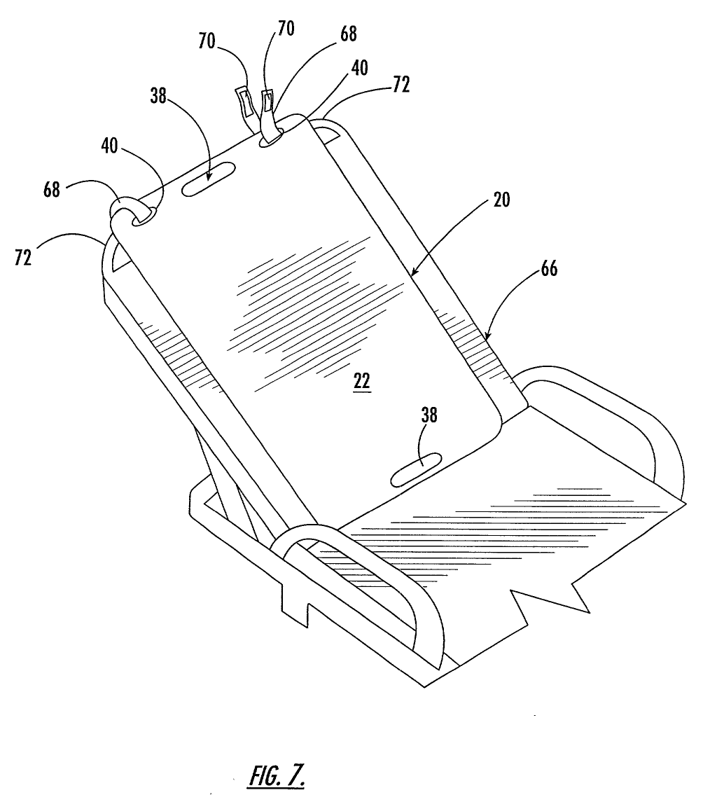

Referring also to FIG. 2 and according to the first embodiment of the present invention, a majority of the marginal portions of the rectangular sheets 22 and 24 are fused together and a majority of the nonmarginal portions of the sheets are not fused together, so that the cooling container 20 has an interior space 26 between portions of the sheets. Referring to FIGS. 4 and 6, sections of adjacent marginal portions of the two sheets 22 and 24 are not fused together, so that an opening 28 to the interior space 26 is defined. In accordance with the first embodiment of the present invention, the opening 28 is advantageously large enough to substantially contemporaneously receive all of the ice or the like to be used in the cooling container 20. For example and as illustrated in FIG. 1, the length L1 of the opening 28 (FIGS. 4 and 6) is preferably at least about one third of the length L2 of the cooling container 20, and more preferably the length L1 of the opening is about one half of the length L2 of the cooling container. Referring to FIG. 4, the opening 28 is manually openable and closeable by a fastening device 30, which is closed in FIG. 4 and open in FIG. 6.

Referring to FIG. 4 and in accordance with the first embodiment, the fastening device 30 includes male and female zipper-like plastic fastening devices 32, 34 that are respectively fused to the adjacent marginal portions of the sheets 22, 24 that define the opening 28. In the example illustrated in FIG. 4, the male fastening device 32 includes three side-by-side, elongate, parallel ridges that are respectively removably received by three side-by-side, elongate, parallel grooves of the female fastening device 34. The ridges and grooves of the fastening device 30 preferably extend uniformly for the entire length of the opening 28. Examples of fastening devices that can be adapted for use in place of the fastening device 30 of the cooling container 20 are disclosed in U.S. Pat. Nos. 5,138,750; 5,140,727; 5,817,380; 5,955,160; 6,021,557; 6,059,457; 6,065,872, each of which is incorporated herein by reference. Other types of fastening devices can also be used.

Referring back to FIG. 1, marginal portions of the sheet 22 that are fused to respective portions of the sheet 24 and the fastening device 30 are designated by the numeral 36. The fastening device 30 is hidden from view in FIG. 1, and is therefore illustrated by broken lines in FIG. 1. Portions of the sheet 22 that are respectively fused to corresponding portions of the sheet 24 that extend around handle holes 38 and attachment holes 40 that extend through the cooling container 20 are respectively designated by numerals 42 and 44. Accordingly, the interior space 26 of the cooling container 20 is substantially hermetically sealed when the opening 28 is completely closed by the fastening device 30. Additionally and in accordance with the first embodiment, another elongated portion of the sheet 22 that is fused to a corresponding elongated portion of the sheet 24 is designated by the numeral 46.

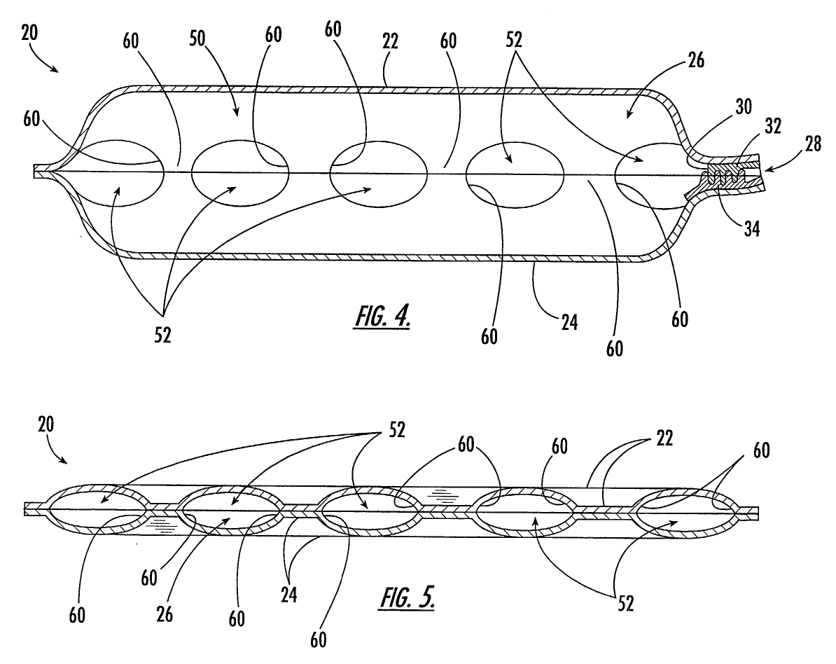

Elongated portions of the sheet 22 are fused to corresponding elongated portions of the sheet 24 so that the interior space 26 of the cooling container 20 is generally separated into sections, and these fused portions are denoted by the numeral 48 in FIG. 1. Referring to FIGS. 3-5, a first section of the interior space 26 is in the form of an internal chamber 50 for holding ice or the like, a second section of the interior space is in the form of internal passages 52 for receiving water originating from the melting ice in the chamber, and a third portion of the interior space is in the form of an internal reservoir 54 for receiving, via the passages, water originating from the melting ice. The passage 52 illustrated in FIG. 3 is representative of each of the other passages and is open solely at its opposite ends, with one of the ends being open to the chamber 50 and the other of the ends being open to the reservoir 54. Referring back to FIG. 1, the internal sections of the interior space 26 are hidden from view, but the approximate boundary between the chamber 50 and ends of the passages 52 is schematically illustrated by a broken line 56, and the approximate boundary between the other ends of the passages and the reservoir 54 is schematically illustrated by a broken line 58.

Referring to FIGS. 3-5 and from the frame of reference of being within the interior space 26 of the cooling container 20, the elongated fused together portions of the sheets 22 and 24 that are designated by numeral 48 in FIG. 1 can be characterized as forming elongated obstructions in the interior space, and portions of the obstructions are designated by the numeral 60. In accordance with an alternative embodiment of the present invention, the one or more obstructions 60 are initially separate components from 30 the sheets 22, 24 that are mounted within the interior space 26 of the cooling container 20. In accordance with the first embodiment, the obstructions 60 are the structure that partially separates the interior space 26 into the sections, namely a section referred to as the chamber 50, another section referred to as the reservoir 54, and a final section, with the obstructions being a portion of the structure that separates this final section into the passages 52.

The cooling container 20 is prepared for use by opening the fastening device 30 so that the opening 28 is opened, and then a bag 62 containing ice, such as a seven pound bag of ice in one example, is placed in the chamber 50 via the opening as shown in FIG. 6, in accordance with the first embodiment of the present invention. Advantageously, portions of the one or more obstructions 60 that are adjacent the chamber 50 function to hold the bag 62 of ice in the chamber. Alternatively, one or more loose pieces of ice or any other cold object(s) are placed into the chamber 50. Thereafter, the opening 28 is closed via the fastening device 30. The bag 62 containing ice preferably has multiple holes 64, so that as the ice melts within the bag, resulting water flows into the passages 50 and reservoir 54.

With the bag 62 of ice in the chamber 50 and water within the passages 52 and reservoir 54, the obstruction(s) 60 preferably advantageously restrict how fat or thick the passages and reservoir can become, so that the chamber is preferably relatively thicker and functions as a pillow-like portion of the cooling container 20, and the passages and reservoir together function as a relatively thinner pad-like portion of the cooling container. As one example of use and referring to FIG. 3, the portions of the cooling container 20 respectively associated with the chamber 50, reservoir 54 and passages 52 respectively define thicknesses T1, T2 and T3, with the thickness T1 preferably being at least about 50% greater than each of the thicknesses T2 and T3, and most preferably the thickness T1 being at least about twice each of the thicknesses T2 and T3. For one size of the cooling container 20, this advantageously allows the pillow-like portion to be used to cool a user's head and neck, and the pad-like portion to be used at the same time to cool at least the user's upper torso. The water in the cooling container 20 recirculates within the interior space 26 in response to movements of the user of the cooling container against and relative to the cooling container. The cooling container 20 can be 30 conveniently carried like a bag, and even be used in a bag-like capacity, by folding it in half and passing a hand through both of the handle holes 38.

Referring to FIG. 7, the attachment holes 40 can be used to attach the cooling container 20 to a structure, such as a seating device that can be in the form of a chair 66, or the like. For example and in one method of use, straps 68 with connectors 70, such as hook and loop connectors sold under the Velcro brand, are passed through the attachment holes 40 and used to attach the cooling container 20 to an upper portion of the back of a chair 66, by passing the straps around a rung 72 of the chair. The cooling container can be used on other types of chairs, or bleachers, or the like. The cooling container 20 may be covered with a towel, or the like, if the user desires to moderate the cooling effect of the cooling container.

A smaller version of the cooling container 20 can be used such that the pillow-like chamber 50 functions as a back support, such as in one example a lumbar support, and the pad-like combination of the passages 52 and reservoir 54 support the user's buttocks. According to any of the above or other examples, a user may place appropriate item(s) to be kept cool in the chamber 50 with the ice or other cooling medium. For example, a flexible beverage container can be placed in the interior space 26, preferably the chamber 50, of the body cooler 20, and a tube can extend from the beverage container to the outside of the cooling container so that a user of the cooling container can suck on the end of the tube that is external of the cooling container to drink the beverage from the beverage container.

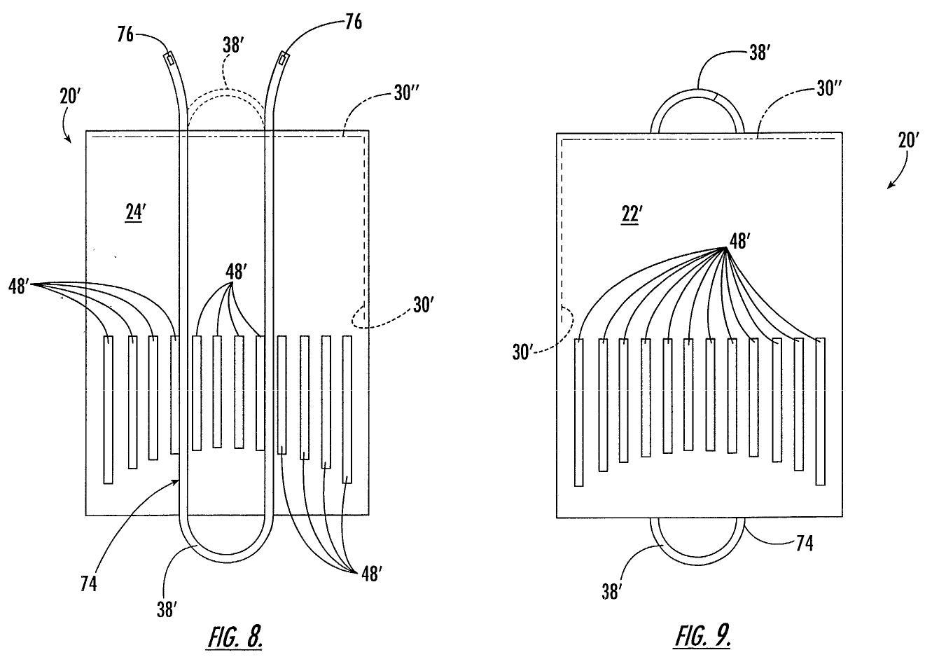

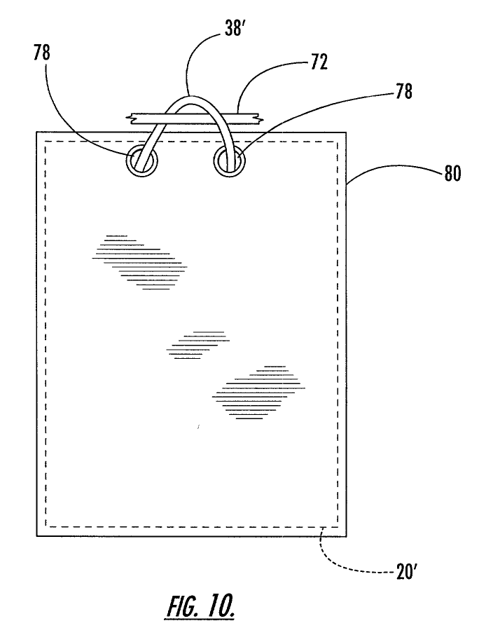

A second embodiment of the present invention will now be described with reference to FIGS. 8-12. The cooling container 20′ of the second embodiment is like the cooling container 20 of the first embodiment, except for variations noted and variations that will be readily apparent to those of ordinary skill in the art in view of this disclosure. In FIGS. 8-9, the opening (for example see the opening 30 of the first embodiment) and its associated fastening device 30′ are hidden from view, but the fastening device 30′ is shown in broken lines along a side edge of the cooling container 20′. Alternatively to the second embodiment, the opening (for example see the opening 30 of the first embodiment) and its associated fastening device 30″ can be along another edge of the cooling container 20′, as indicated by the broken-line illustration of the alternatively positioned fastening device 30″ in FIGS. 8-9.

In accordance with the second embodiment, the cooling container 20′ includes external handles 38′ that are defined by a strip 74 externally attached to the body of the cooling container, rather than including integral handles as in the first embodiment. The ends of the strip 74 that form one of the handles 38′ can advantageously be disconnected and reconnected to one another by attachment devices 76, such as hook and loop connectors sold under the Velcro brand. For example, FIG. 8 illustrates the upper one of the handles 38′ in its open state in solid lines and in its closed state in broken lines, and the same handle is illustrated in its closed state in FIG. 9.

Referring to FIG. 10, with the upper handle 38′ in the open state illustrated in FIG. 8, the free end portions of the handle can be passed through grommet-reinforced openings 78, or the like, in a cloth cover 80, or the like, and around a rung 72 of a chair, or the like, to secure the cooling container 20′ to the cloth cover and the chair. A majority of the cooling container 20′ is hidden from view behind the cover 80 in FIG. 10, and therefore the cooling container is partially illustrated by broken lines in FIG. 10.

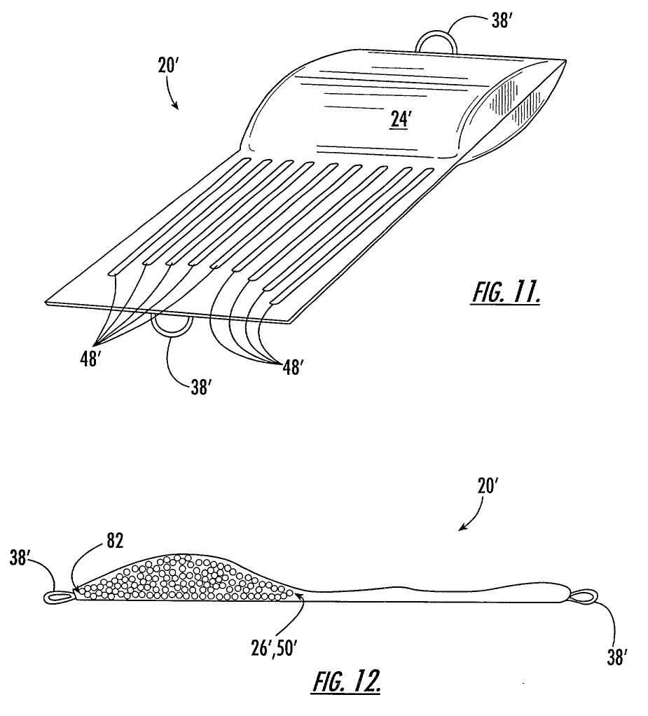

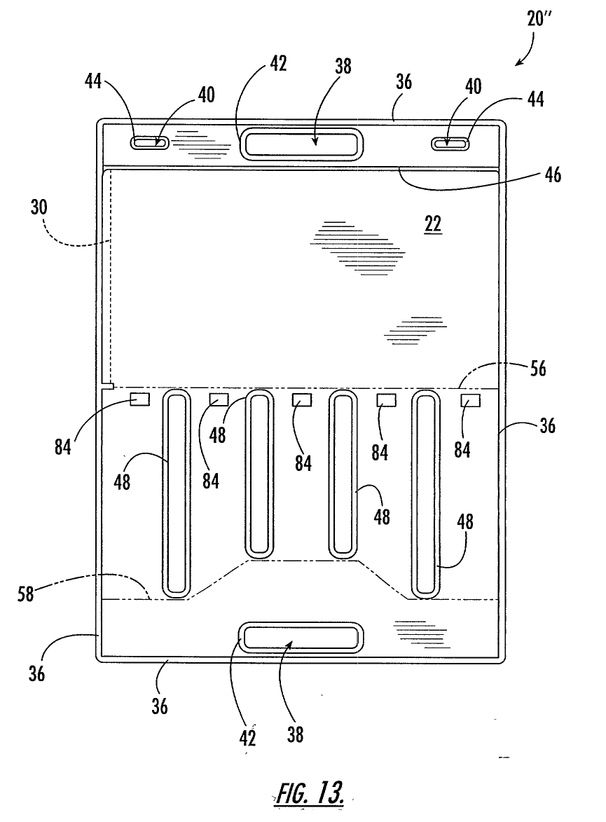

FIGS. 11-12 illustrate the cooling container 20′ with its chamber 50′ at least partially filled with ice 82, according to the second embodiment of the present invention.

FIG. 13 illustrates a cooling container 20″ in accordance with a third embodiment of the present invention. The cooling container 20″ of the third embodiment is identical to the cooling container 20 of the first embodiment, except that it further includes supplemental obstructions resulting from additional nonmarginal portions of the sheets 22 and 24 being bonded together, preferably by fusing. The additional nonmarginal portions of the sheet 22 that are fused to respective portions of the sheet 24 are designated by the numeral 84. The supplemental obstructions resulting from the fused portions identified by numeral 84 are positioned in open ends of the passages 52 and function to further restrict ice from traveling from the chamber 50 into the passages.

In accordance with an alternative embodiment of the present invention, a tube has an internal end within the reservoir 54 and an external end that is exterior to the cooling container and equipped with a spray nozzle. In accordance with this alternative embodiment, a user of the cooling container can force cool water within the reservoir 54 to be sprayed from the spray nozzle and onto their body for cooling purposes.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A container for receiving ice and being used to cool one or more users, comprising:
a body constructed of flexible and water-impermeable material, defining an interior space and an opening to the interior space through which the ice can be introduced into the interior space, and including walls that are positioned on opposite sides of the interior space, wherein portions of the walls are connected to define at least one obstruction positioned in the interior space for partially separating first and second sections of the interior space, the first section is adjacent the opening for receiving the ice, and the first section and the second section are connected so that water resulting from the ice can flow from the first section to the second section; and
a fastening device for closing the opening so that the interior space is substantially sealed.

2. A container according to claim 1, wherein the fastening device is a zipper-type fastening device.

3. A container according to claim 1, wherein the body includes opposite ends and a length is defined between the opposite ends, and the opening includes opposite ends and a length is defined between the opposite ends of the opening, and the length of the opening is at least about one third of the length of the body.

4. A container according to claim 1, wherein elongated portions of the walls are connected together to define the obstruction, so that the obstruction is elongate.

5. A container according to claim 1, wherein portions of the walls are connected to define a plurality of obstructions spaced apart from one another in the interior space and partially separating the first section from the second section.

6. A container according to claim 5, wherein the obstructions are positioned in the second section.

7. A container according to claim 5, wherein the obstructions are positioned in a third section of the interior space that is between the first section and the second section.

8. A container according to claim 5, wherein the obstructions are elongated and define a plurality of passages.

9. A container according to claim 8, wherein each of the obstructions has opposite first and second ends, the first ends of the obstructions together partially separate the first section from the second section, and each of the passages has an end open to the first section.

10. A container according to claim 8, wherein the obstructions are positioned in the second section.

11. A container according to claim 8, wherein the obstructions are positioned in a third section of the interior space that is between the first section and the second section, each of the obstructions includes opposite ends with one of the ends being adjacent the first section and the other of the ends being adjacent the second section and each of the passages has opposite ends with one of the ends open to the first section and the other of the ends open to the second section so that the passages provide communication paths between the first and second sections.

12. A container according to claim 1, further comprising at least one handle connected to or formed in the body.

13. A container according to claim 1, in combination with a seating device and at least one strap mounting the container to the seating device.

14. A container according to claim 1, in combination with a bag that contains ice and is positioned in the first section, with a plurality of apertures defined in the bag so that water resulting from the ice can flow from the first section to the second section, and wherein the obstruction holds the bag in the first section.

15. A container according to claim 1, wherein the walls include first and second polymeric laminates that are fused together to form the body of the container.

16. A container for receiving ice and being used to cool one or more users, comprising:
first and second sheets arranged one on top of the other, with each sheet having a margin extending around the periphery of the sheet and the margin including first and second sections, wherein the first section of the margin of the first sheet is connected to the first section of the margin of the second sheet so that an interior space is defined between nonmarginal portions of the sheets, an opening to the interior space is defined between the second section of the margin of the first sheet and the second section of the margin of the second sheet, at least one nonmarginal portion of the first sheet is connected to at least one nonmarginal portion of the second sheet to define at least one obstruction positioned in the interior space for at least partially separating first and second sections of the interior space, the first section is adjacent the opening for receiving the ice, and the first and second sections are connected so that water resulting from the ice can flow from the first to the second section; and
a fastening device having first and second parts respectively mounted to the second section of the margin of the first sheet and the second section of the margin of the first sheet, wherein the first and second parts of the fastening device are for being mated to one another to close the opening, so that the interior space is substantially sealed, and for being separated from one another to open the opening.

17. A container according to claim 16, wherein the body includes opposite ends and defines a length between its opposite ends, and the opening includes opposite ends and defines a length between its opposite ends, and the length of the opening is at least about one third of the length of the body.

18. A container according to claim 16, wherein the first part of the fastening device includes an elongated ridge and the second part of the fastening device includes an elongated groove, and the parts of the fastening device are mated and releasably interlocked by forcing the ridge into the groove.

19. A container according to claim 16, wherein the first and second sheets are each polymeric laminates, the first section of the margin of the first sheet is fused to the first section of the margin of the second sheet, the nonmarginal portion of the first sheet is fused to the nonmarginal portion of the second sheet, the first part of the fastening device is fused to the second section of the margin of the first sheet, and the second part of the fastening device is fused to the second section of the margin of the second sheet.

20. A container according to claim 16, wherein portions of the sheets are connected to define a plurality of obstructions spaced apart from one another in the interior space and partially separating the first section from the second section.

21. A container according to claim 20, wherein the obstructions are positioned in the second section.

22. A container according to claim 20, wherein the obstructions are positioned in a third section of the interior space that is between the first section and the second section.

23. A method of cooling a user, comprising:
placing ice into an interior space of a container so that an obstruction within the interior space holds the ice in a first section of the interior space and allows water melting from the ice to flow into a second section of the interior space, with the placing including
placing a sufficient amount of ice in the first section so that the portion of the container containing the first section is thicker than the portion of the container containing the second section; placing the user's head on the portion of the container containing the first section so that the portion of the container containing the first section functions as a pillow and the user's head is cooled by the ice in the pillow; and
placing at least the user's upper torso on the portion of the container containing the second section so that the portion of the container containing the second section functions as a pad and at least the user's upper torso is cooled by the water in the pad.
